(19) United States
(12) Patent Application Publication
(10) Pub. No.: US 2012/0004245 A1
(43) Pub. Date: Jan. 5, 2012

(54) COMPOUNDS FOR THE TREATMENT OF POSTERIOR SEGMENT DISORDERS AND DISEASES

(75) Inventors: Jesse A. MAY, Fort Worth, TX (US); David P. BINGAMAN, Weatherford, TX (US); Carmelo ROMANO, Fort Worth, TX (US)

(73) Assignee: ALCON RESEARCH, LTD., Fort Worth, TX (US)

(21) Appl. No.: 13/168,271

(22) Filed: Jun. 24, 2011

Related U.S. Application Data
(60) Provisional application No. 61/361,003, filed on Jul. 2, 2010.

(51) Int. Cl. A61K 31/519; A61K 31/416; A61P 9/10; A61P 9/00; A61P 27/02; A61K 31/437; A61K 31/4365

(52) U.S. Cl. 514/260.1; 514/303; 514/407; 514/301

(57) ABSTRACT

The use of certain urea compounds, for the treatment of retinal disorders associated with pathologic ocular angiogenesis and/or neovascularization is disclosed.

This application claims priority under 35 U.S.C. §119 to U.S. Provisional Patent Application No. 61/361,003 filed Jul. 2, 2010, the entire contents of which are incorporated herein by reference.

The present invention relates to the use of compounds for the treatment of the exudative and non-exudative forms of age-related macular degeneration, diabetic retinopathy, and retinal edema, and other diseases involving pathologic ocular angiogenesis and/or vascular permeability.

### BACKGROUND OF THE INVENTION

AMD is the most common cause of functional blindness in individuals over the age of 50 in industrialized countries and a common cause of unavoidable blindness worldwide. The vision loss associated with AMD typically occurs only at the most advanced stages of the disease, when patients progress from nonexudative (“dry”) AMD to either exudative AMD with choroidal neovascularization (CNV) or to geographic atrophy. Although only 10% to 20% of all nonexudative AMD patients will progress to exudative AMD, this form of AMD accounts for 80-90% of the functional vision loss associated with this disorder. Exudative AMD, also termed neovascular or wet AMD, is characterized by the growth of pathologic CNV into the subretinal space. The CNV has a tendency to leak blood and fluid, causing symptoms such as scotoma and metamorphopsia, and is often accompanied by the proliferation of fibrous tissue. Invasion of this fibrovascular membrane into the macula can induce photoreceptor degeneration resulting in progressive, severe and irreversible vision loss. Without treatment, most affected eyes will have poor central vision (≦20/200) within 2 years.

Another blinding retinal disorder known as proliferative diabetic retinopathy (PDR) is also characterized by pathologic posterior segment neovascularization (PSNV). PDR is the most common cause of legal blindness in patients with diabetes mellitus and is characterized by pathologic preretinal NV. Moreover, in patients with diabetes mellitus, diabetic macular edema (DME) is the major cause of vision impairment overall. Diabetes mellitus is characterized by persistent hyperglycemia that produces reversible and irreversible pathologic changes within the microvasculature of various organs. Diabetic retinopathy (DR), therefore, is a retinal microvascular disease that is manifested as a cascade of stages with increasing levels of severity and worsening prognoses for vision.

Nonproliferative diabetic retinopathy (NPDR) and subsequent macular edema are associated, in part, with retinal ischemia that results from the retinal microvasculopathy induced by persistent hyperglycemia. NPDR encompasses a range of clinical subcategories which include initial “background” DR, where small multifocal changes are observed within the retina (e.g., microaneurysms, “dot-blot” hemorrhages, and nerve fiber layer infarcts), through preproliferative DR, which immediately precedes the development of PNV. The histopathologic hallmarks of NPDR are retinal microaneurysms, capillary basement membrane thickening, endothelial cell and pericyte loss, and eventual capillary occlusion leading to regional ischemia. Data accumulated from animal models and empirical human studies show that retinal ischemia is often associated with increased local levels of proinflammatory and/or proangiogenic growth factors and cytokines, such as vascular endothelial growth factor (VEGF), prostaglandin E2, insulin-like growth factor-1 (IGF-1), Angiopoietin 2, etc. Diabetic macular edema can be seen during either NPDR or PDR. However, it often is observed in the latter stages of NPDR and is a prognostic indicator of progression towards development of the most severe stage, PDR, where the term “proliferative” refers to the presence of preretinal neovascularization as previously stated.

Pathologic ocular angiogenesis, including PSNV, is known to occur as a cascade of events that progress from an initiating stimulus to the formation of abnormal new capillaries. While the specific inciting cause(s) of PSNV in both exudative AMD and PDR is still unknown, the elaboration of various proangiogenic growth factors appears to be a common stimulus. Soluble growth factors, such as vascular endothelial growth factor (VEGF), platelet-derived growth factor (PDGF), basic fibroblast growth factor (bFGF or FGF-2), insulin-like growth factor 1 (IGF-1), angiopoietins, etc., have been found in tissues and fluids removed from patients with pathologic ocular angiogenesis. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. Differentiation into a mature capillary is denoted by the presence of a continuous basement membrane and normal endothelial junctions between other endothelial cells and vascular-supporting cells called pericytes; however, this differentiation process is often impaired during pathologic conditions. More specifically, increased levels of PDGF appear to play a role in the maturation of new blood vessels by acting as a survival factor for pericytes.

Until recently, patients with vision-threatening PSNV had limited treatment options. Many of the approved therapies, such as focal laser photocoagulation for extrafoveal CNV and photodynamic therapy with Visudyne® for Exudative AMD, were often palliative and could be associated with vision-threatening complications themselves. For example, grid or panretinal laser photocoagulation and surgical interventions, such as vitrectomy and removal of preretinal membranes, are the only options currently available for patients with PDR. However, the approval of intravitreal anti-VEGF therapies has revolutionized the treatment of pathologic PSNV, specifically Exudative AMD.

Substantial evidence suggests that the soluble growth factor, vascular endothelial growth factor-A (VEGF-A), plays a critical role in the pathogenesis of PSNV. The VEGFs (VEGF-A, -B, -C, -D, -E and placenta growth factor [PlGF]), are a family of homodimeric glycoproteins that bind with varying affinities to their cell surface receptors, VEGF receptor 1 (VEGFR1), VEGFR2, and VEGFR3. VEGF-A, commonly referred to as VEGF, is a dimeric 36-46 kDa glycosylated protein with an N-terminal signal sequence and a heparin binding domain. Six different pro-angiogenic splice variants of VEGF have been identified; these differ in their number of amino acids and include VEGF₂₀₆, VEGF₁₈₉, VEGF₁₈₃, VEGF₁₆₅, VEGF₁₄₅, and VEGF₁₂₁. The shorter forms are more freely diffusible, e.g., VEGF₁₂₁ is completely devoid of the heparin-binding domain, and VEGF₁₆₅ is the most abundant of these lower molecular weight variants. The larger variants, VEGF₂₀₆ and VEGF₁₈₉, are matrix-bound and unlikely to bind to endothelial cell receptors.

VEGF is the most extensively characterized ligand of VEGFR-1 and VEGFR-2, which are cell membrane receptors primarily located on the surface of vascular endothelial cells and exhibit intrinsic tyrosine kinase activity following ligand binding. These two VEGF receptor tyrosine kinases (RTKs) are major contributors to vascular morphogenesis and pathological neovascularization through two primary mechanisms: (1) stimulation of new vessel growth (vasculogenesis and/or angiogenesis) and (2) increased vascular hyperpermeability. VEGF, VEGFR1, and VEGFR2 have been localized in ocular fluids and neovascular membranes obtained from patients with neovascular AMD and diabetic retinopathy; perhaps more importantly, the presence of these proteins was associated with increased severity of disease.

The anti-VEGF agents that have been approved for the treatment of neovascular AMD are the ribonucleic acid aptamer, Macugen®, (pegaptanib, Eyetech/OSI/Pfizer) which specifically binds VEGF-A₁₆₅ and Lucentis® (ranibizumab, Genentech/Novartis) an Fab fragment of a humanized monoclonal antibody that binds all isoforms of VEGF-A. Although Macugen® was approved in 2004, patients treated with intravitreal Macugen® in Phase III studies continued to experience vision loss during the first year of treatment, although the rate of vision decline in the Macugen®-treated group was slower than the rate in the sham-treated group. Macugen® was less effective during the second treatment year than during the first year, demonstrating benefit in only one of these two pivotal studies.

In contrast, intravitreal Lucentis®, approved in 2006, administered at 4 week intervals in Phase III trials maintained best-corrected visual acuity (BCVA) in 95% of treated patients and improved BCVA by 15 or more letters in 24 to 40% of treated patients. These notable benefits were sustained over the 24 month treatment duration when injecting Lucentis every month. However, when Lucentis® was administered at 12-week intervals following three initial monthly loading doses in patients with Exudative AMD and followed for 12-months, Lucentis® treatment preserved but did not improve visual acuity. Although intravitreal Lucentis® represents a marked improvement in therapeutic outcomes for patients with neovascular AMD, these and other less favorable results when using dosing frequencies of less than one injection per month suggest that a major unmet medical need of current anti-VEGF therapy is duration-of-action.

A variety of other anti-VEGF strategies are or have been investigated in human clinical trials for Exudative AMD and/or DME such as intravitreal Avastin® (bevacizumab, Genentech), a full-length humanized monoclonal antibody against VEGF-A that was approved in 2004 for intravenous treatment of colorectal cancer; intravitreal VEGF TrapR₁R₂ (Regeneron) a 110 kDa, recombinant chimeric protein comprising portions of the extracellular, ligand-binding domains of the human VEGFR1 and VEGFR2 fused to the Fc portion of human IgG and binds all isoforms of VEGF-A as well as placental growth factor (PlGF); the combination therapy of intravitreal Lucentis® plus an anti-PDGF aptamer (Ophthotech), in an attempt to induce NV regression through simultaneous blockade of active ECs and pericytes; as well as local or systemic delivery of various receptor tyrosine kinase inhibitors (RTKi's)

Receptor tyrosine kinase inhibitors (RTKi's) are a newer class of anti-angiogenic compounds that block VEGF signal transduction by inhibiting the intrinsic tyrosine phosphorylation of the cell membrane receptors. RTKi's are being clinically evaluated for both ophthalmic and non-ophthalmic indications. A significant advantage for the use of RTKi's in the treatment of angiogenesis-dependent diseases is their potential to provide a more complete blockade of VEGF signaling by blocking receptor activation from multiple ligands. Moreover, because the most effective RTKi's simultaneously block multiple signaling pathways, they are anticipated to provide advantages in efficacy over current therapies directed at a solitary growth factor. As small molecules (<500 Da), RTKi's have the potential for enhanced inter- and intracellular distribution and are more amenable to formulation within sustained delivery devices when compared to large biological molecules, such as antibodies or large peptides.

Related to ophthalmic indications, an increasing body of scientific evidence suggests that RTKi's may provide substantial advantages in the treatment of pathologic PSNV and/or retinal edema. PKC412 (CGP41251, Novartis), an RTKi selective against PKC isoforms as well as VEGFRs and PDGFRs, provided partial reductions in enhanced foveal thickness as measured by OCT and an improvement in visual acuity following oral administration in patients with existing DME. However, gastrointestinal adverse events, such as diarrhea, nausea, and vomiting, and increased transaminase activity were dose-limiting. Oral administration of another RTKi, PTK787 (vatalanib, Novartis and Schering AG), has undergone clinical investigation in patients with neovascular AMD. PTK787 is a more selective VEGFR inhibitor compared to PKC412 and has been shown to provide significant inhibition of PSNV in rodent models. Although results from the Phase 1/2 neovascular AMD study have not been released, the most common adverse events reported from published Phase 1/2 oncology studies using oral daily dosing of PTK787 has been fatigue, nausea, dizziness, vomiting, anorexia, and diarrhea. Recently, the RTKi, Pazopanib (GlaxoSmithKline) has entered into clinical trials for exudative AMD using topical ocular administration.

An effective locally-delivered selective RTKi against pathologic ocular angiogenesis, PSNV, exudative AMD, DME, retinal/macular edema, DR, and retinal ischemia, would provide substantial benefit to the patient through inhibition and/or regression of angiogenesis and inhibition of increased vascular permeability, thereby significantly maintaining or improving visual acuity. Effective treatment of these pathologies would improve the patient's quality of life and productivity within society. Also, societal costs associated with providing assistance and health care to the visually impaired could be dramatically reduced.

### SUMMARY OF THE INVENTION

This application is directed to the use of certain urea compounds to treat persons suffering from posterior segment disorders associated with pathologic ocular angiogenesis/neovascularization and/or retinal edema, including the exudative and non-exudative forms of AMD, diabetic retinopathy, which includes preproliferative diabetic retinopathy (collectively DR), DME, and PDR, retinal or macular edema, central or branch retinal vein occlusion, and ischemic retinopathies.

### DETAILED DESCRIPTION OF THE INVENTION

Posterior segment neovascularization is the vision-threatening pathology responsible for the two most common causes of acquired blindness in developed countries: exudative age-related macular degeneration (AMD) and proliferative diabetic retinopathy (PDR).

In addition to changes in the retinal microvasculature induced by hyperglycemia in diabetic patients leading to macular edema, proliferation of neovascular membranes is also associated with vascular leakage and edema of the retina. Where edema involves the macula, visual acuity worsens. In diabetic retinopathy, macular edema is the major cause of vision loss. Like angiogenic disorders, laser photocoagulation is used to stabilize or resolve the edematous condition. While reducing further development of edema, laser photocoagulation is a cytodestructive procedure, that, unfortunately will alter the visual field of the affected eye.

An effective pharmacologic therapy for ocular NV and edema would likely provide substantial efficacy to the patient, in many diseases thereby avoiding invasive surgical or damaging laser procedures. Effective treatment of the NV and edema would improve the patient's quality of life and productivity within society. Also, societal costs associated with providing assistance and health care to the blind could be dramatically reduced.

The present invention is based, in part, on the discovery that certain urea compounds that inhibit receptor tyrosine kinases are useful for the treatment of AMD, DR, DME, retinal/macular edema, ischemic retinopathies, and disease associated with posterior segment neovascularization (PSNV). An effective locally-delivered selective RTKi would provide substantial benefit to the patient through inhibition and/or regression of angiogenesis and inhibition of increased vascular permeability, thereby significantly maintaining or improving visual acuity. Considering the well described list of untoward side-effects associated with systemic anti-VEGF therapy in oncology, such as hypertension, nephrotic syndrome, thromboembolic events, bleeding, gastrointestinal perforations, voice changes, mucosal toxicity, hand-foot syndrome, fatigue, neurological complications (e.g., reversible posterior leukoencephalopathy syndrome), myelosuppression, and transaminase elevations, coupled with the observation of some of these adverse reactions in early ophthalmic trials following systemic dosing of anti-VEGF compounds, local ocular delivery of a selective RTKi may provide unique treatment advantages in both safety and efficacy for patients with debilitating posterior segment disease. In addition, these compounds have been shown to provide regression of PSNV in animal models, a pharmacologic characteristic not found when using inhibitors that block only the VEGF pathway, such as intravitreal Lucentis®. Therefore, the present invention may provide clinical benefit in one or more of three major areas: increased efficacy, increased duration of action, and reduced systemic side-effects.

The preferred compounds for use in the methods of the present invention are compounds I through VII set forth below:

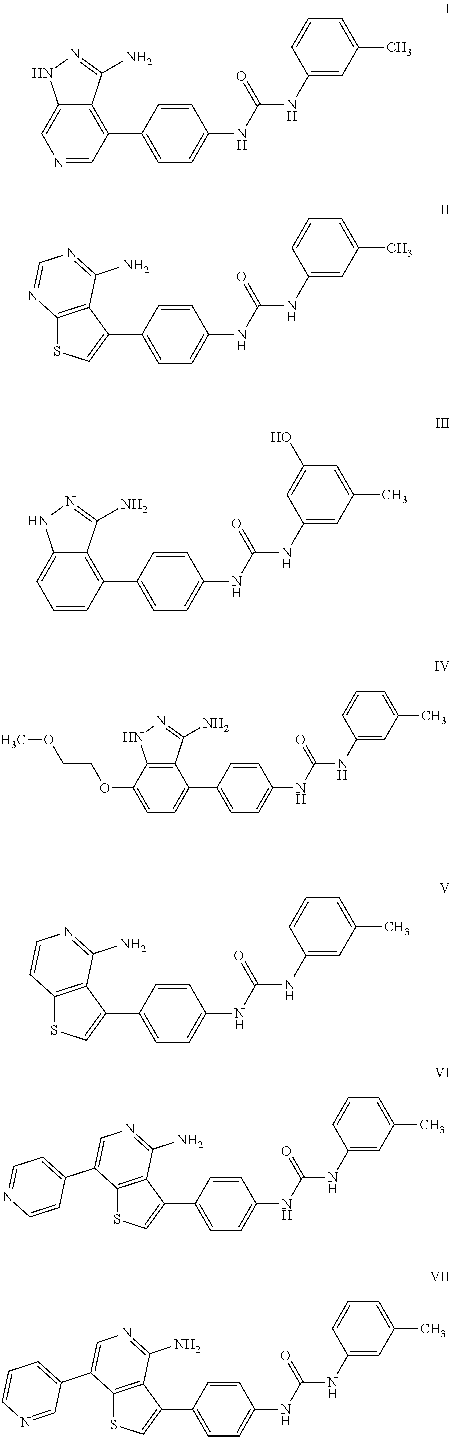

Chemical names for Compounds I-VII are set forth in Table 1, below:

| Compound No. | Compound Name |
|---|---|
| I | 1-[4-(3-Amino-1H-pyrazolo[3,4-c]pyridin- |
| | 4-yl)-phenyl]-3-m-tolyl-urea |
| II | 1-[4-(4-Amino-thieno[2,3-d]pyrimidin-5- |
| | yl)-phenyl]-3-m-tolyl-urea |
| III | 1-[4-(3-Amino-1H-indazol-4-yl)-phenyl]-3- |
| | (3-hydroxy-5-methyl-phenyl)-urea |
| IV | 1-{4-[3-Amino-7-(2-methoxy-ethoxy)-1H- |
| | indazol-4-yl]-phenyl}-3-m-tolyl-urea |
| V | 1-[4-(4-Amino-thieno[3,2-c]pyridin-3-yl)- |
| | phenyl]-3-m-tolyl-urea |
| VI | 1-[4-(4-Amino-7-pyridin-4-yl-thieno[3,2- |
| | c]pyridin-3-yl)-phenyl]-3-m-tolyl-urea |
| VII | 1-[4-(4-Amino-7-pyridin-3-yl-thieno[3,2- |
| | c]pyridin-3-yl)-phenyl]-3-m-tolyl-urea |

Compounds I-VII of the present invention are known, and their syntheses are disclosed in U.S. application serial no. 2006/0178378 (Compound I), U.S. application serial no. 2003/0181468 (Compound II), U.S. Pat. No. 7,297,709 (Compounds III and IV), and U.S. application serial nos. 2005/0020619 and 2005/0026944 (Compounds V-VII), each of which is herein incorporated by reference. In addition, two other related urea compounds (VIII and IX) that are known (see structures shown below) and their syntheses are disclosed in U.S. Pat. No. 7,297,709 and were shown to be ineffective in the following pharmacology studies.

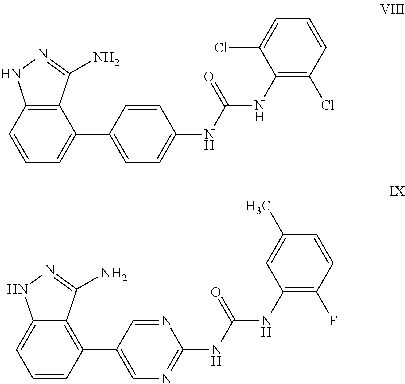

It is also contemplated that pharmaceutically acceptable salts of any of compounds I through VII, and any combination of compounds I-VII may be used in the methods of the present invention.

As used herein, the terms “pharmaceutically acceptable salt” means any anion of Compounds I-VII that would be suitable for therapeutic administration to a patient by any conventional means without significant deleterious health consequences. Examples of preferred pharmaceutically acceptable anions, or salts, include chloride, bromide, acetate, benzoate, maleate, fumarate, and succinate.

The Compounds disclosed herein may be contained in various types of pharmaceutical compositions, in accordance with formulation techniques known to those skilled in the art. The pharmaceutical compositions containing the Compounds described herein may be administered via any viable delivery method or route, however, local administration to the eye is preferred. It is contemplated that all local routes to the eye may be used including topical, subconjunctival, periocular, retrobulbar, subtenon, intracameral, intravitreal, intraocular, subretinal, and suprachoroidal administration. Systemic or parenteral administration may be feasible including but not limited to intravenous, subcutaneous, and oral delivery. The most preferred method of administration will be intravitreal or subtenon injection of solutions or suspensions, or intravitreal or subtenon placement of bioerodible or non-bioerodible devices, or by topical ocular administration of solutions or suspensions, or posterior juxtascleral administration of a gel formulation. Another preferred method of delivery is intravitreal administration of a bioerodible implant administered through a device such as that described in US application publication number 2007/0060887.

The present invention is also directed to the provision of compositions adapted for treatment of retinal and optic nerve head tissues. The ophthalmic compositions of the present invention will include one or more of the described Compounds I-VII and a pharmaceutically acceptable vehicle. Various types of vehicles may be used. The vehicles will generally be aqueous in nature. Aqueous solutions are generally preferred, based on ease of formulation, as well as a patient's ability to easily administer such compositions by means of instilling one to two drops of the solutions in the affected eyes. However, the compounds for use in the present invention may also be readily incorporated into other types of compositions, such as suspensions, viscous or semi-viscous gels, or other types of solid or semi-solid compositions. Suspensions may be preferred for compounds that are relatively insoluble in water. The ophthalmic compositions of the present invention may also include various other ingredients, such as buffers, preservatives, co-solvents, and viscosity building agents.

An appropriate buffer system (e.g., sodium phosphate, sodium acetate or sodium borate) may be added to prevent pH drift under storage conditions.

Ophthalmic products are typically packaged in multidose form. Preservatives are thus required to prevent microbial contamination during use. Suitable preservatives include: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% weight/volume (“% w/v”).

The route of administration (e.g., topical, ocular injection, parenteral, or oral) and the dosage regimen will be determined by skilled clinicians, based on factors such as the exact nature of the condition being treated, the severity of the condition, and the age and general physical condition of the patient.

In general, the doses used for the above described purposes will vary, but will be in an effective amount to prevent or treat AMD, DR, and retinal edema. As used herein, the term “pharmaceutically effective amount” refers to an amount of one or more of the compounds described herein which will effectively treat AMD, DR, and/or retinal edema in a human patient. The doses used for any of the above-described purposes will generally be from about 0.01 to about 100 milligrams per kilogram of body weight (mg/kg), administered one to four times per day. When the compositions are dosed topically, they will generally be in a concentration range of from 0.001 to about 10% w/v, with 1-2 drops administered 1-4 times per day.

As used herein, the term “pharmaceutically acceptable carrier” refers to any formulation that is safe, and provides the appropriate delivery for the desired route of administration of an effective amount of at least one compound of the present invention.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The present invention is based on the discovery that urea compounds that block tyrosine autophosphorylation could be selected out of various genera by using a series of efficacy pharmacology assays to demonstrate their intrinsic ability to (1) inhibit retinal and choroidal neovascularization; (2) cause regression of retinal and choroidal neovascularization; and (3) block retinal vascular permeability. In addition the same pharmacology assays were used to show that other urea compounds from the same genera did not possess the same intrinsic efficacy properties. Therefore, the pharmacologic properties discovered for these urea molecules were previously unknown. The results of the various urea compounds in the selected assays are summarized in the table below. The inventor(s) was/were personally involved in the design and analysis of all studies mentioned below.

### Example 1

### KDR Assay

METHODS. A 7-point HTRF (Homogeneous Time Resolved Fluorescence) kinase assays were performed using a Biomek 3000 Robotic Workstation in a 96-well plate format to determine IC₅₀ values for the test compounds for KDR (VEGFR2) kinase using KinEASE-TK kit from CisBio. This is a general kit for tyrosine kinases including KDR kinase. The KDR kinase was purchased from Cell Signaling Technology. The assay is run in two steps. The phosphorylation of the biotin-tagged generic peptide substrate (2 mM) is initiated by the addition of ATP (10 mM) in the presence of KDR kinase (5 ng in 50 ml reaction mixture) in step 1 and the reaction is stopped after 30 min incubation at room temperature by the addition of a mixture containing two HTRF detection reagents and EDTA in step 2. The substrate, enzyme, and ATP dilutions were made with the buffer provided by CisBio. Compound dilutions were made either in 5% DMSO or 10:10, (DMSO:Ethanol) to prepare 4× working stock solutions. The HTRF detection reagents were an antibody to phosphotyrosine, labeled with Eu(K) (the HTRF donor), and a streptavidin-XL665 (the HTRF acceptor). The resulting HTRF signal (ratio of 665 nm/620 nm) is measured using Tecan HTRF plate reader and data were analyzed using a non-linear, iterative, sigmoidal-fit computer program (OriginPro 8.0) to generate the inhibition constants for the test compounds.

RESULTS. Seven unique, structurally-dissimilar, small molecule inhibitors of receptor tyrosine kinases (RTKi's) (Compounds I-VII) demonstrated substantial potency in two in vitro assays, including significant efficacy against VEGF-induced proliferation in a cellular assay. Specifically, all RTKi's demonstrated an IC50<1 nM when tested for activity against KDR (human VEGFR2) in an enzyme-based assay, as described herein (Table 2). In addition, the two other related urea compounds (VIII and IX, Table 2) were shown to essentially have no activity against KDR as compared to Compounds I-VII.

TABLE 2

| | | VEGF ind | Relative |
|---|---|---|---|
| Compound | KDR | BREC prolif | Potency vs |
| No. | IC₅₀ nM | EC₅₀ nM | Reference |
| I | <1 | 0.16 | 6.4 ± 3.4 |
| II | <1 | 0.11 | 14.5 ± 15.1 |
| III | <1 | 0.45 ± 0.05 | 0.565 ± 0.205 |
| IV | <1 | 0.10 ± 0.01 | 5.78 ± 1.24 |
| V | <1 | 1.57 ± 1.24 | 0.775 ± 0.262 |
| VI | <1 | 0.07 ± 0.069 | 8.0 ± 2.6 |
| VII | <1 | 0.11 ± 0.086 | 4.0 ± 1.1 |
| VIII | >10,000 | Not Active | N/A |
| IX | >10,000 | Not Active | N/A |

### Example 2

### BREC Assay

METHODS. Because of their ability to potently inhibit VEGFR2, each Compound I-VII was evaluated for activity against VEGF-induced proliferation of bovine retinal endothelial cells (BRECs). Bovine retinal endothelial cells are seeded at 3000-7000 cells/well in fibronectin-coated 96 well plates in MCDB-131 growth media with 10% FBS. After 24 hours the growth media is replaced with MCDB-131 media supplemented with 1% FBS, glutamine, heparin, hydrocortisone, and antibiotics. After another 22-24 hours the cells are treated with or without 50 ng/ml VEGF media and the test compounds in the 1% FBS media. After 30 hours BrdU is then added for the final 16 hours of the incubation. All cells are then fixed and assayed with a colorimetric BrdU ELISA kit.

RESULTS. All Compounds (I-VII) demonstrated potent and efficacious inhibition of VEGF-induced proliferation, where all seven Compounds provided an EC₅₀<2 nM, and six of seven Compounds had an EC₅₀<0.5 nM (Table 2). Moreover, all seven Compounds exhibited a relative potency ≧0.5 of a reference standard RTKi known to provide reproducible efficacy in animal models of posterior segment disease (Table 2). In addition, the two other related urea compounds (VIII and IX, Table 2) were shown to be completely inactive against VEGF-induced proliferation as compared to Compounds I-VII. Because of their inactivity in both the KDR assay and BREC proliferation assay, Compounds VIII and IX were not moved forward for in vivo testing.

### Example 3

### Intravitreal Delivery of Compounds I-VII, Inhibits VEGF-Induced Retinal Vascular Permeability in the Rat

METHODS: Adult Sprague-Dawley rats were anesthetized with intramuscular ketamine/xylazine and their pupils dilated with topical cycloplegics. Rats were randomly assigned to intravitreal injection groups of 0% 0.3%, 1.0%, and 3.0% formulations of Compounds I-VII and a positive control. Ten μl of each compound was intravitreally injected in each treatment eye (n=5˜6 animals per group). Three days following first intravitreal injection, all animals received an intravitreal injection of 10 μl 500 ng hr VEGF in both eyes. Twenty-four hours post-injection of VEGF, intravenous infusion of 3% Evans blue dye was performed in all animals, where 50 mg/kg of Evans blue dye was injected via the lateral tail vein during general anesthesia. After the dye had circulated for 90 minutes, the rats were euthanized. The rats were then systemically perfused with balanced salt solution, and then both eyes of each rat were immediately enucleated and the retinas harvested using a surgical microscope. After measurement of the retinal wet weight, the Evans blue dye was extracted by placing the retina in a 0.2 ml formamide (Sigma) and then the homogenized and ultracentrifuged. Blood samples were centrifuged and the plasma diluted 100 fold in formamide. For both retina and plasma samples, 60 μl of supernatant was used to measure the Evans blue dye absorbance (ABS) with at 620/740 nm. The blood-retinal barrier breakdown and subsequent retinal vascular permeability as measured by dye absorbance were calculated as means+/−s.e.m. of net ABS/wet weight/plasma ABS. One way ANOVA was used to determine an overall difference between treatment means. And a test or Man-Whitney rank sum test was performed for a pair-wise comparison between treatment groups, where P<0.05 was considered significant.

RESULTS: In the rat VEGF model, each compound was tested initially using a single ivt injection of either 0.1% or 1% suspension. Six of seven Compounds demonstrated the ability to inhibit VEGF-induced RVP, where five of six Compounds provided >70% inhibition (*P<0.05), at one or more doses as compared to vehicle-injected controls (Table 3). Then each compound was tested in a dose-response manner using a single ivt injection (Table 4).

TABLE 3

| Compound | Efficacy | Efficacy |
|---|---|---|
| No. | (0.1%) | (1%) |
| I | 65.4%* | 88.9%* |
| II | 85.2%* | 85.7%* |
| IV | 73.9%* | −148.2% |
| III | −96.7% | −316.9% |
| V | 64.0% | 40.4% |
| VI | 106.5%* | 70.6%* |
| VII | 84%* | 73.5%* |

TABLE 4

| | | ED₅₀ | | |
|---|---|---|---|---|
| AL# | MW | (nmole) | ED₅₀ (μg) | Potency |
| I | 358.5 | 4.04\hat{ } | 1.447 | 1.2* |
| II | 375.5 | 7.62 | 2.86 | 0.392* |
| III | 373.4 | >80.3 | >30 | 0.028 |
| V | 374.5 | 3.8\hat{ } | 1.42 | 1.38* |
| VI | 451.6 | 1.42 | 0.64 | 2.303* |
| VI | 431.5 | 22.3 | 9.63 | 0.147 |
| VII | 451.6 | 5.47 | 2.47 | 0.621* |

*Compounds are equipotent to reference standard, since 95% confidence limits (CL) encompass 1.0 (LL < 1.0 < UL)

### Example 4

### Prevention and Regression of Preretinal Neovascularization Following Intravitreal Delivery of Compounds I-VII, in the Rat Model of Oxygen-Induced Retinopathy

METHODS: Pregnant Sprague-Dawley rats were received at 14 days gestation and subsequently gave birth on Day 22±1 of gestation. Immediately following parturition, pups were pooled and randomized into separate litters (n=17 pups/litter), placed into separate shoebox cages inside oxygen delivery chamber, and subjected to an oxygen-exposure profile from Day 0-14 postpartum. Litters were then placed into room air from Day 14/0 through Day 14/6 (days 14-20 postpartum). For prevention studies, each pup was randomly assigned into various treatment groups on Day 14/0. For those randomized into an injection treatment group: one eye received a 5 μl intravitreal injection of between 0.01%-1% of a RTKi and the contralateral eye received a 5 μl intravitreal injection of vehicle. At Day 14/6 (20 days postpartum), all animals were euthanized. For regression studies, each pup was randomly assigned as an oxygen-exposed control or into various treatment groups on Day 18/0. For those randomized into an injection treatment group: one eye received a 5 μl intravitreal injection of between 0.01%-1% RTKi and the contralateral eye received a 5 μl intravitreal injection of vehicle. At Day 14/7 (21 days postpartum), all animals were euthanized.

Immediately following euthanasia, retinas from all rat pups were harvested, fixed in 10% neutral buffered formalin for 24 hours, subjected to ADPase staining, and fixed onto slides as whole mounts. Digital images were acquired from each retinal flat mount that was adequately prepared. Computerized image analysis was used to obtain a NV clockhour score from each readable sample. Each clockhour out of 12 total per retina was assessed for the presence or absence of preretinal NV. Statistical comparisons using median scores for NV clockhours from each treatment group were utilized in nonparametric analyses. Each noninjected pup represented one NV score by taking the average value of both eyes and was used in comparisons against each dosage group. Because the pups were randomly assigned and no difference was observed between oxygen-exposed control pups from all litters, the NV scores were combined for all treatment groups. P≦0.05 was considered statistically significant.

RESULTS: In the rat OIR model, each Compound was tested initially using a single ivt injection of either 0.1% or 1% suspension in a prevention paradigm. Six of seven Compounds provided 100% inhibition (P<0.05) at the 1% dose when compared to vehicle (Table 5). Subsequent dose-response prevention studies using a single ivt injection of suspension showed that all seven Compounds were approximately ≧2× more potent against preretinal neovascularization than a reference standard RTKi known to provide reproducible efficacy in the rat OIR model (Table 6). In addition, four of seven compounds were tested in dose-response regression, i.e., intervention, studies using a single ivt injection of suspension showed that all four Compounds were near 2× more potent at regressing preretinal neovascularization versus the reference RTKi (Tables 7).

TABLE 5

| | Efficacy | Efficacy |
|---|---|---|
| | (0.1%) | (1%) |
| Compound | Median- | Median- |
| No. | value | value |
| I | 100%* | 100%* |
| II | 32.2 | 100%* |
| IV | 100%* | 100%* |
| III | 1.5% | 100%* |
| V | 16.9 | 100%* |
| VI | 35.7 | 88.4* |
| VII | 9.6 | 100* |

TABLE 6
Rat OIR Prevention

| Compound | MW | ED₅₀ (nmole) | ED₅₀ (μg) | Potency |
|---|---|---|---|---|
| I | 358.5 | 13.44 | 4.82 | 5.75 |
| II | 375.5 | 12.17 | 4.57 | 6.23 |
| III | 373.4 | 15.85 | 5.92 | 4.03 |
| IV | 431.5 | 13.23 | 5.71 | 4.2 |
| V | 374.5 | 24.11 | 9.03 | 1.94 |
| VI | 451.6 | 13.31 | 6.01 | 3.86 |
| VII | 451.6 | 15.74 | 7.11 | 3.23 |
| Reference | 375.4 | 71.04 | 26.67 | 1 |

TABLE 7
Rat OIR Regression

| Compound | ED₅₀ (nmole) | ED₅₀ (μg) | Potency |
|---|---|---|---|
| I | 25.47 | 9.13 | 2.26 |
| I | 25.47 | 9.13 | 2.26 |
| II | 24.31 | 9.13 | 2.3 |
| III | — | — | — |
| IV | — | — | — |
| V | — | — | — |
| VI | 39.46 | 17.82 | 1.75 |
| VII | 20.86 | 9.42 | 2.49 |
| Reference | 64.57 | 24.24 | 1 |

### Example 5

### Prevention and Regression of Laser-Induced Choroidal Neovascularization (CNV) Following a Intravitreal Delivery of Compounds I-VII, in the Mouse

METHODS. CNV was generated by laser-induced rupture of Bruch's membrane. Briefly, 4 to 5 week old C57BL/6J mice were anesthetized using intraperitoneal administration of ketamine hydrochloride (100 mg/kg) and xylazine (5 mg/kg) and the pupils of both eyes dilated with topical ocular instillation of 1% tropicamide and 2.5% MYDFIN®. One drop of topical cellulose (GONIOSCOPIC®) was used to lubricate the cornea. A hand-held cover slip was applied to the cornea and used as a contact lens to aid visualization of the fundus. Three to four retinal burns were placed in randomly assigned eye (right or left eye for each mouse) using the Alcon 532 nm EyeLite laser with a slit lamp delivery system. The laser burns were used to generate a rupture in Bruch's membrane, which was indicated ophthalmoscopically by the formation of a bubble under the retina. Only mice with laser burns that produced three bubbles per eye were included in the study. Burns were typically placed at the 3, 6, 9 or 12 o'clock positions in the posterior pole of the retina, avoiding the branch retinal arteries and veins.

Each mouse was randomly assigned into one of the following treatment groups: noninjected controls, sham-injected controls, vehicle-injected mice, or one of three Compound-injected groups. Control mice received laser photocoagulation in both eyes, where one eye received a sham injection, i.e. a pars plana needle puncture. For intravitreal-injected animals, one laser-treated eye received a 2 or 5 μl intravitreal injection of 0.1%-3% of a RTKi or vehicle. For prevention studies, the intravitreal injection was performed immediately after laser photocoagulation. For the regression, i.e., intervention, study with RTKi's, the intravitreal injection was performed at Day 7 after laser photocoagulation and a group of lasered, non-injected mice were also harvested at Day 7 for controls. At 14 days post-laser, all mice were anesthetized and systemically perfused with fluorescein-labeled dextran. Eyes were then harvested and prepared as choroidal flat mounts with the RPE side oriented towards the observer. All choroidal flat mounts were examined using a fluorescent microscope. Digital images of the CNV were captured, where the CNV was identified as areas of hyperfluorescence within the pigmented background. Computerized image analysis was used to delineate and measure the two dimensional area of the hyperfluorescent CNV per lesion (μm²) for the outcome measurement. The median CNV area/burn per mouse per treatment group or the mean CNV area/burn per treatment group was used for statistical analysis depending on the normality of data distribution; P<0.05 was considered significant.

RESULTS. In pilot prevention studies in the mouse CNV model, two of the Compounds tested to date caused a notable reduction in laser-induced CNV following a single ivt injection of doses ranging from 0.1-1.0% suspension. Two of three compounds provided statistically significant inhibition at the highest dose tested when compared to vehicle-injected controls (Table 8).

The results of using a single intravitreal (ivt) injection of Compound I and II.

Subsequent dose-response prevention studies using a single ivt injection of suspension showed that Compound I was more potent, while Compound II was slightly less potent than the reference RTKi in inhibiting CNV formation (Table 9). In the regression study, Compound I was equivalent to the reference RTKi in causing the regression of existing CNV when administered via single ivt injection at Day 7 post laser; and Compound II also demonstrated significant CNV regression effect (57.4%, Table 9)

TABLE 8
Mouse CNV Studies: initial efficacy (Prevention)

| Compound No. | Prevention Efficacy |
|---|---|
| I | 2 μg/0.1% = −53.8% 20 μg/1.0% = 37.6% |
| II | Pilot |
| | 2 μg/0.1% = 40.6% |
| | 20 μg/1.0% = 69.0%* |
| IV | 2 μg/0.1% = 1.1% |
| | 6 μg/0.3% = 13.9% |
| | 20 μg/1.0% = 49.7%* |

TABLE 9
Mouse CNV Studies: Prevention and Regression
CNV

| | | Prevention | |
|---|---|---|---|
| Compound | MW | Potency | Regression Potency |
| I | 358.5 | 5.9 | 2.64* |
| II | 375.5 | 0.76# | NA |
| | | | (no dose response study was done, |
| | | | but showed 57.5% regression at |
| | | | 3%-60 μg) |
| Reference | 375.4 | 1 | 1 |

*Compounds are equipotent to reference standard, since 95% confidence limits (CL) encompass 1.0 (LL < 1.0 < UL)
#Approximate potency number, since the lines are not parallel.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

We claim:

1. A method for treating posterior segment neovascularation, AMD, DR, and/or retinal edema in a patient which comprises administering to the patient in need of such treatment an ophthalmic composition comprising a therapeutically effective amount of at least one compound selected from the group consisting of
1-[4-(3-Amino-1H-pyrazolo[3,4-c]pyridin-4-yl)-phenyl]-3-m-tolyl-urea
1-[4-(4-Amino-thieno[2,3-d]pyrimidin-5-yl)-phenyl]-3-m-tolyl-urea
1-[4-(3-Amino-1H-indazol-4-yl)-phenyl]-3-(3-hydroxy-5-methyl-phenyl)-urea
1-{4-[3-Amino-7-(2-methoxy-ethoxy)-1H-indazol-4-yl]-phenyl}-3-m-tolyl-urea
1-[4-(4-Amino-thieno[3,2-c]pyridin-3-yl)-phenyl]-3-m-tolyl-urea
1-[4-(4-Amino-7-pyridin-4-yl-thieno[3,2-c]pyridin-3-yl)-phenyl]-3-m-tolyl-urea
1-[4-(4-Amino-7-pyridin-3-yl-thieno[3,2-c]pyridin-3-yl)-phenyl]-3-m-tolyl-urea,
and pharmaceutically acceptable salts thereof.

2. The method of claim 1, wherein the compound is 1-[4-(4-Amino-thieno[2,3-d]pyrimidin-5-yl)-phenyl]-3-m-tolyl-urea.

3. The method of claim 1, wherein the concentration of said compound in the ophthalmic composition is from 0.001% to 10%.

4. The method of claim 3, wherein the concentration of said compound in the ophthalmic composition is 1%.

5. The method of claim 1, wherein the ophthalmic composition is administered via a route selected from the group consisting of topical, subconjunctival administration, periocular administration, retrobulbar administration, subtenon administration, intracameral injection, intravitreal injection, intraocular injection, subretinal administration, suprachoroidal administration and posterior juxtascleral administration.

6. The method of claim 5, wherein the ophthalmic composition is administered via intravitreal injection.

7. A method for causing regression of ocular neovascularization, said method comprising administering to a patient in need thereof an ophthalmic composition comprising a therapeutically effective amount of at least one compound selected from the group consisting of 1-[4-(3-Amino-1H-pyrazolo[3,4-c]pyridin-4-yl)-phenyl]-3-m-tolyl-urea
1-[4-(4-Amino-thieno[2,3-d]pyrimidin-5-yl)-phenyl]-3-m-tolyl-urea
1-[4-(3-Amino-1H-indazol-4-yl)-phenyl]-3-(3-hydroxy-5-methyl-phenyl)-urea
1-{4-[3-Amino-7-(2-methoxy-ethoxy)-1H-indazol-4-yl]-phenyl}-3-m-tolyl-urea
1-[4-(4-Amino-thieno[3,2-c]pyridin-3-yl)-phenyl]-3-m-tolyl-urea
1-[4-(4-Amino-7-pyridin-4-yl-thieno[3,2-c]pyridin-3-yl)-phenyl]-3-m-tolyl-urea
1-[4-(4-Amino-7-pyridin-3-yl-thieno[3,2-c]pyridin-3-yl)-phenyl]-3-m-tolyl-urea, and
pharmaceutically acceptable salts thereof.

8. The method of claim 7, wherein the compound is 1-[4-(4-Amino-thieno[2,3-d]pyrimidin-5-yl)-phenyl]-3-m-tolyl-urea.

9. The method of claim 7, wherein the concentration of said compound in the ophthalmic composition is from 0.001% to 10%.

10. The method of claim 9, wherein the concentration of said compound in the ophthalmic composition is 1%.

11. The method of claim 7, wherein the ophthalmic composition is administered via a route selected from the group consisting of topical, subconjunctival administration, periocular administration, retrobulbar administration, subtenon injection, intracameral administration, intravitreal injection, intraocular injection, subretinal administration, suprachoroidal administration and posterior juxtascleral administration.

12. The method of claim 11, wherein the ophthalmic composition is administered via intravitreal injection.
